(19) United States
(12) Patent Application Publication
(10) Pub. No.: US 2020/0245881 A1
(43) Pub. Date: Aug. 6, 2020

(54) BIOCOMPATIBLE MICROELECTRODE

(71) Applicant: ATATÜRK ÜNIVERSITESI BILIMSEL ARASTIRMA PROJELERI BIRIMI, Erzurum (TR)

(72) Inventors: Ahmet HACIMÜFTÜOGLU, Erzurum (TR); Ibrahim Yücel ÖZBEK, Erzurum (TR); Erdal SÖNMEZ, Erzurum (TR); Bülent ÇAVUSOGLU, Erzurum (TR); Ali TAGHIZADEHGHALEHJOUGHI, Erzurum (TR); Mehmet ERTUGRUL, Erzurum (TR); Hasan TÜRKEZ, Erzurum (TR); Fazil HACIMÜFTÜOGLU, Erzurum (TR); Merve ACAR, Erzurum (TR); Zekeriyya ERDEMIR, Erzurum (TR); Duygu EKINCI, Erzurum (TR)

(21) Appl. No.: 16/639,673

(22) PCT Filed: Aug. 14, 2018
(86) PCT No.: PCT/TR2018/050432

§ 371 Date: Feb. 17, 2020

(30) Foreign Application Priority Data
Aug. 23, 2017 (TR) 2017/12611

(51) Int. Cl. A61B 5/04

(52) U.S. Cl. CPC A61B 5/04001; A61B 2562/0209

(57) ABSTRACT

A biocompatible microelectrode for in vivo and/or in vitro instant monitoring of the neurotransmitter release and reuptake for brain diseases and behavior disorders, which has a recording surface located on the end portion that contacts the neurotransmitter and a conductive circuit which is positioned on the holding portion which enables holding the biocompatible microelectrode during application, and which transfers the measurement data taken from the recording surface to sensors/measurement device. Selective and precise in vivo and in vitro measurement of the neurotransmitter is enabled by coating the isolated recording surface of the biocompatible microelectrode with a biocompatible surface coating.

### TECHNICAL FIELD

The invention is related to a biocompatible microelectrode used for in vivo and in vitro instant imaging of the neurotransmitter release and reuptake in brain diseases and behavior disorders.

The invention is particularly related to a biocompatible microelectrode which can maintain its measurement stability after several uses both in vivo and in vitro by means of its surface coating.

### PRIOR ART

Voltammetry is the generic name for the measurement method used for the techniques based on a current with the administration of continuously changing data through electrodes. The microelectrodes used in this technique have different designs, while they have certain disadvantages for the user when they are required to be co-administered. The most important one among them is that the coatings such as Nafion and MPD (Membrane Potential Dye) have very low durability and very short in vivo and in vitro lifetime; furthermore they are not biocompatible. None of the electrodes commonly available in the market, used for subsecond neurotransmitter measurements with such coatings, are biocompatible.

The earthing inlet is one of the critical details which define the detection precisions of such types of electrodes. As the earthing inlet is located outside of the electrode of existing electrodes, the noise developing at the background may prevent obtaining the actual measurement result. Furthermore, as keeping it in hand is difficult due to its structure, the electrode may frequently fall and break. These electrodes also lose their measurement precision when they are requested to be used again after the initial use. The detection precisions are also inadequate for identifying low current signals.

The application WO2017048421 in the previous art is related with methods and systems for near-simultaneous measurement of neuron activity and neurotransmitter concentration. The system uses voltammetry probe for neurotransmitter recordings and an electrophysiology probe for neural ensemble recordings. However, said system does not mention an electrode configuration which eliminates the defects of the present art. Furthermore, it does not include a biocompatible barrier. This barrier enables selective measuring of the desired neurotransmitter among tens of items that can be measured in the brain. Furthermore, the barrier is biocompatible. This barrier is exclusive for our invention and it does not have any other examples.

After all, a development was required in the related technical field due to the negative aspects and inadequacy of the existing solutions described so far.

### OBJECTIVE OF THE INVENTION

The present invention eliminates the foregoing disadvantages and it is related to a biocompatible microelectrode to introduce new advantages to the related technical field.

The main objective of the invention conducts in vivo and in vitro neurotransmitter measurement for use in voltammetry technique, and suggests a biocompatible microelectrode for instant monitoring. The recording zones of the electrode were covered with a wide platinum surface and they provided ease in neurotransmitter detection. The neurotransmitters can be measured at lower concentrations compared to the existing electrodes.

Another objective of the invention is to measure with a high detection precision and without being affected from in vitro sounds.

Another objective of the invention is to facilitate handling without damage thanks to the wide silicone base it comprises. The electrode has been protected against being broken with small impacts thanks to the physical properties of the photo-resist and the substrate used. These properties have enabled production at dimensions which will not pathologically harm but also which will not easily break (both in vivo and in vitro).

Another objective of the invention is to eliminate the noise received from the background during the measurement, thanks to the earthing inlet placed at the central area.

Another objective of the invention is that it can stably work without losing precision in long term (such as 1 month) reuses without being affected from in vivo and in vitro conditions by means of AgNPs/graphene oxide, 2-aminobenzoimidasole, 2-aminobenzothiazole type of coatings on it. As a result of this coating, the neurotransmitter intended to be measured can be measured with selective, biocompatible and specific barriers.

The structure and characteristics of the invention and its all advantages will more clearly be understood with reference to the following figures, and therefore, the invention is required to be understood by taking into consideration the attached figures together with the following detailed description.

### BRIEF DESCRIPTION OF THE DRAWINGS

FIG. 1 is top view of the invented biocompatible microelectrode.

FIG. 2 is detailed view of the end portion of the invented biocompatible microelectrode.

FIG. 3 is detailed view of the holding portion of the invented biocompatible microelectrode.

FIG. 4 shows detailed views of the different types of the invented biocompatible microelectrode.

The drawings are not necessarily scaled and the details that are not essential to understand the present invention may have been omitted. Apart from that, the elements which are at least substantially identical or have at least substantially identical functions are shown with the same reference number.

### REFERENCE NUMBERS

1. Biocompatible microelectrode
A. End portion
   10. Ceramic base
   20. Recording surface
B. Holding portion
   30. Silicone base
   40. Conductor circuit
C. Earthing inlet

### DETAILED DESCRIPTION OF THE INVENTION

The preferred embodiments of the invention are described herein with certain embodiments only to facilitate understanding the invention and without having any restrictive effect.

The invention is related with a biocompatible microelectrode (1) for in vivo and/or in vitro instant monitoring of the neurotransmitter release and reuptake for brain diseases and behavior disorders, which comprises a recording surface (20) located on the end portion (A) that contacts the neurotransmitter and a conductive circuit (40) which is positioned on the holding portion (B) which enables holding the biocompatible microelectrode (1) during application, and which transfers the measurement data taken from the recording surface (20) to sensors/measurement device. Selective and precise in vivo and in vitro measurement of the neurotransmitter is enabled by coating a biocompatible microelectrode (1) surface coating on the isolated recording surface (20) of the biocompatible microelectrode.

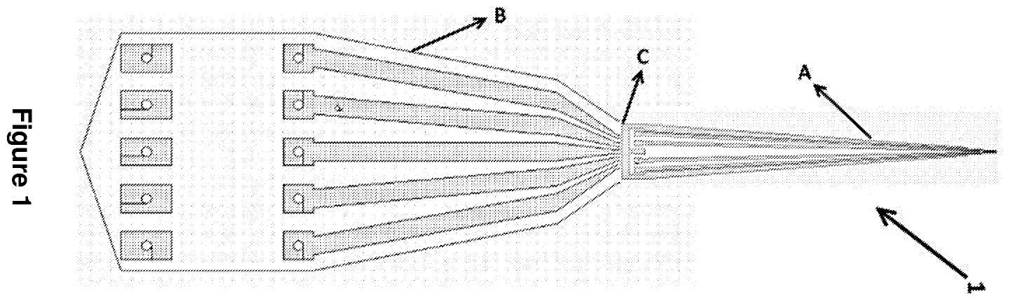

FIG. 1 is the top view of the invented biocompatible microelectrode. According to this figure, the invention substantially comprises an end portion (A) which obtains the measurement results, and a holding portion (B) which enables holding the device during application. These two main elements are connected to each other with an earthing inlet (C). Thanks to positioning the earthing inlet (C) of the earthing connection on the electrode, in the middle of the end portion (A) and the holding portion (B), the level of impacting the measurement results by preventing the noise realized at the background during measurement has been reduced.

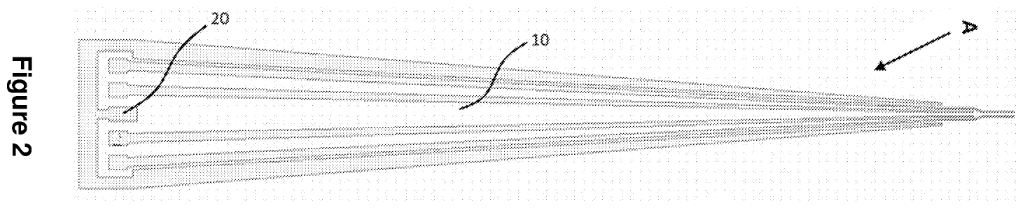

FIG. 2 is the detail view (A) of the end portion (A) of the invented biocompatible microelectrode. Accordingly, the ceramic base (10) of the end portion (A) constitutes the main structure. The preferably platinum recording surface (20) which enables realizing the recording operation is located on the ceramic base (10) that enables adhesion to the surface and enabling isolation. The ceramic base (10) that constitutes the end portion (A) base has a biocompatible and non-toxic structure.

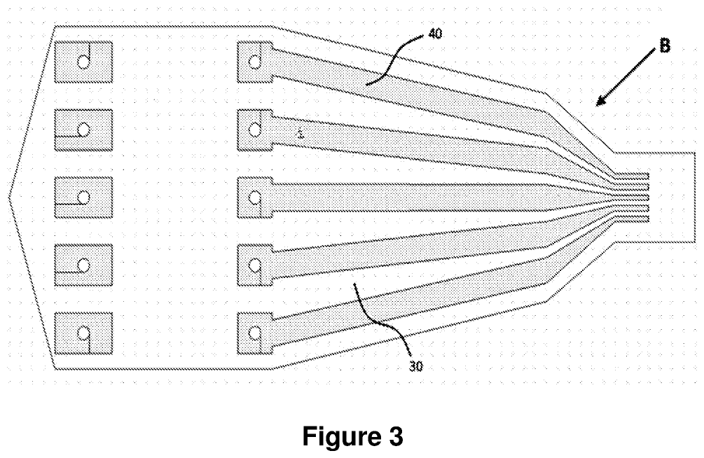

FIG. 3 is the detail view of the holding portion (B) of the invented biocompatible microelectrode (1). The holding portion (B) comprises a silicon base (30) in a form to prevent falling and breaking of the electrode during application by facilitating its being held in hand. The copper conductor circuit (40) positioned on the silicon base (30) which constitutes the base of the holding portion (B) enables moving the measurement results received from the end point (A) to the device. Copper conductive circuit (40) is attached by printing on a wide base made of silicate and plastic.

The device detects the currents at nA level, and sends them to the analysis software of the computer wirelessly after the reinforcement and digitization operations. The device is portable the dimensions of 0.6 cm×3.7 cm×2.4 cm and 7.1 gr weight, and briefly realizes the following operations.

After connecting to the biocompatible microelectrode (1), the current is created in proportion to the neurotransmitter concentration in the environment of the biocompatible microelectrode (1) with 0.7 V activation energy received from the device. This current at nanoampere levels is sent to the device's intensifier. The read current values are filtered and digitized, and then transferred through the microcontroller to the analysis interface prepared with computer software by Bluetooth. It is analyzed with analysis algorithms written on this interface.

For surface coating, AgNPs/graphene oxide (silver nanoparticle added graphene oxide) is used on the recording surface (20). For selectivity, barriers were created with 2-aminobenzoimidasole, 2-aminobenzothiasole derivatives according to the type of neurotransmitter, and biocompatible, selective, precise electrodes were obtained. This surface coating can maintain its measurement stability after several uses both in vivo and in vitro thanks to its surface coating.

Biocompatible positive photoresist and negative photoresist with high breakdown voltages and compatible dielectric coefficients suitable for high resolution fabrication were used for the isolation of the microelectrodes as the isolation layer/substance on the recording surface (20).

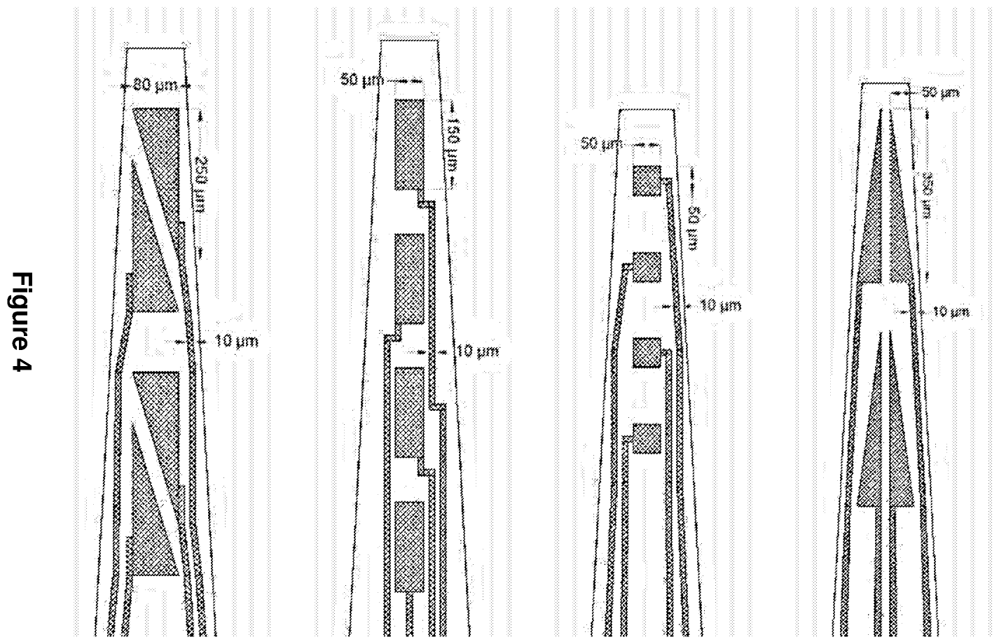

Different forms of biocompatible microelectrodes (1) in FIG. 4 are the illustrations of different embodiments of the invention. Many embodiments may be created according to the foregoing descriptions in many different forms and combinations.

1. A biocompatible microelectrode used for instant in vitro and in vivo imaging of the neurotransmitter release and reuptake in brain diseases and behavior disorders, comprising;
a recording surface obtaining the measurement data and located on an end portion that contacts the neurotransmitter,
a conductive circuit positioned on a holding portion that enables holding the biocompatible microelectrode during application, transferring data received from a recording surface to sensors/measurement device for measurement,
the biocompatible microelectrode characterized by comprising;
a biocompatible surface coating coated on the isolated recording surface to enable selective and precise in vivo and in vitro measurement of the neurotransmitter.

2. The biocompatible microelectrode according to claim 1, comprising an earthing inlet positioned between the end portion and the holding portion, on which the earthing is connected to prevent noise during measurement.

3. The biocompatible microelectrode according to claim 1, wherein the recording surface isolation material includes positive and negative photoresist.

4. The biocompatible microelectrode according to claim 1, wherein the biocompatible surface coating is AgNPs/graphene oxide, 2-aminobenzoimidasole or 2-aminobenzothiasole.

5. The biocompatible microelectrode according to claim 1, comprising a silicone base which constitutes the base of the holding portion, enabling holding the electrode in hand during application and protecting it against falling and breaking.

6. The biocompatible microelectrode according to claim 1, comprising a ceramic base which provides surface adhesion and isolation, and forming the base of the end portion.

7. The biocompatible microelectrode according to claim 1, wherein the recording surface is made of platinum.

8. The biocompatible microelectrode according to claim 1, wherein the conductive circuit is made of copper.
